(19) United States
(12) Patent Application Publication
(10) Pub. No.: US 2012/0045824 A1
(43) Pub. Date: Feb. 23, 2012

(54) Device for early diagnostics of cancer

(76) Inventor: Lev Tovberg, Hadera (IL)

(21) Appl. No.: 13/136,350

(22) Filed: Aug. 1, 2011

(30) Foreign Application Priority Data
Aug. 17, 2010 (IL) 207661

(51) Int. Cl. C12M 1/38

(52) U.S. Cl. 435/286.1

(57) ABSTRACT

Method of cancer treatment with direct contact between proteins secreted by cancer tumor and patient's blood T-lymphocytes including the stage of weakening during some time humoral system of immunity by medical preparation, in particular by immunodepressant against this system, without weakening cellular system of immunity.

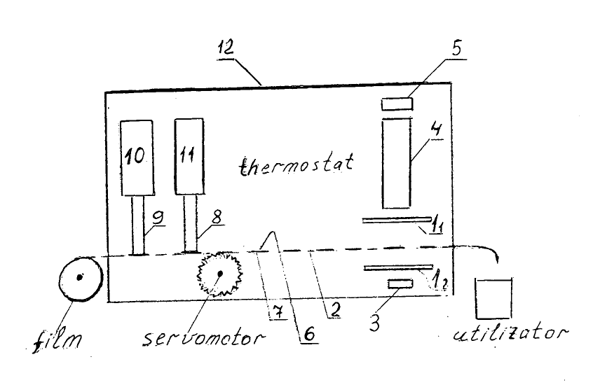

### BACKGROUND OF INVENTION

The invention relates to the field of diagnosing new formations in human organism, in particular to locating malignant tumors (cancer).

For more than a century the mankind purposefully fights one of the most dangerous diseases—cancer of different organs.

Up to the present time a great number of attempts have been made in treating cancer, and positive results have been obtained in many cases, but a universal (general) method of treatment or approach to treatment has not yet been found. Though there exist many methods for treating separate kinds of cancer—cancer of definite organs.

Hereby, it has been stated long ago that the patient who was diagnosed with cancer at an early stage, ideally at the initial stage, has more chances for being cured.

Several methods and technical means for diagnosing new formations in human organism have been known and are used (or can be used) in a number of cases, in particular with use of ultrasonic, infrasonic and sonic waves, also fluorescence emissions—see, for example, the corresponding patents in patent libraries (i.e. storages of inventions) in a number of leading countries, in particular according to International classification and specifically in Int. Cl. A61B 8/08, as well as US patents in accordance with US. Cl: U.S. Pat. No. 4,657,851 dated 1984, U.S. Pat. No. 5,270,788 dated 1993, U.S. Pat. No. 5,552,292 dated 1994, U.S. Pat. No. 5,260,186 dated 1991, U.S. Pat. No. 4,871,661 dated 1985, U.S. Pat. No. 7,655,411 dated Feb. 02, 2010.

But nearly all the known methods and devices for malignant tumors diagnosing cannot for sure determine oncological disease at the initial stage that could have become the basis of opportune treatment and, as a far-going goal—full recovery of the patient.

Up to the present time there exists only one, officially recognized by the State, method of cancer early diagnostics realized by laboratory “Oncolab” (USA) characterizing a special blood analysis (see patent U.S. Pat. No. 5,629,286 dated 1996) which is certified by US FDA and by governmental “Medicare”.

This method of blood analysis “AMAS” (Malignin Antibody in serum) is based on the fact that all kinds of cancer have a common (one and the same) protein called Malignin. Even in small concentrations it boosts the immune system for producing antibodies against this protein. These antibodies of the type IgM can be easily recognized in blood, and it is possible to get information about the cancer in the organism due to the presence of these antibodies.

Laboratory “Oncolab” is the only one in the world which carried out such diagnostics.

But the method mentioned is also characterized by essential disadvantages:

necessity of sending each blood sample to the USA under condition of deep freezing. That's why the cost of checking is rather high (e.g., with delivery from Israel the cost of checking one blood sample equals to $350), and the time of checking practically attains 3 weeks,

impossibility of mass application of this method by other countries and health protection units in them due to high cost of the necessary equipment,

this method (according to medical data) is not universal for mass diagnostics.

That's why the method mentioned cannot serve as the basis for prophylactic medical examinations for the country's population.

The aim of the proposed invention consists in excluding the disadvantages of the solution mentioned. The author's opinion on the advantages of the invention proposed was not shaken by the last information from Israeli press (Russian language newspaper “Epoch”, Sharon region, issue 761 from 30.06.2010, p. 6 “Israeli medicine in the Ukraine”) on opening an Israeli Medical Center in the Ukraine rendering services on cancer diagnostics and prophylaxis. (www.ccuit lissod.com).

### SUMMARY OF THE INVENTION

The aim set forth (early diagnostics of oncological disease irrespective of the kind of disease and the body organ targeted) is attained by means of a proposed device which characterizes the breakthrough in the field studied.

The device is based on different reaction of blood lymphocytes during the contact with phytohaemagglutinin (T-lymphocytes stimulator) with people having or not having an oncological disease.

The device consists of two polarization filters in the form of plates embracing a transparent element for positioning an organic substance sample for studying (a mixture of small portions of blood lymphocyte samples with phytohaemagglutinin), positioned parallel and with mutually perpendicular direction of polarization vectors between a light source and a microscope ocular with photoelement at the exit.

Hereby the element for sample positioning serves as a unit for delivering the said mixture of patient's blood lymphocytes with phytohaemagglutinin to a zone of polarization filters during 21-25 minutes starting with the moment of the said samples contact with phytohaemagglutinin which comes from its delivery unit into each blood lymphocytes sample at the entrance of the unit for delivering blood lymphocyte sample studied.

In this case polarization filters, unit for blood lymphocyte samples delivery, unit for phytohaemagglutinin delivery and capacity for blood lymphocyte samples mixture with phytohaemagglutinin are placed into a thermostat with working temperature of 36.5-37.5 ° C. for preventing blood coagulation.

When light goes through polarization filters (plates) in the microscope and is not fixed by the photoelement, i.e. when lymphocytes do not manifest the properties of polarized light depolarization, the presence of oncological disease is stated.

### BRIEF DESCRIPTION OF THE DRAWING

The invention is further explained by a drawing where a schematic representation of the proposed device for diagnosing an oncological disease is given.

### DETAILED DESCRIPTION OF THE PREFERRED EMBODIMENT

The proposed device for early diagnostics of oncological disease characterizing a breakthrough in the field studied, as it has been mentioned in section “summary of the invention”, is based on different reaction of blood lymphocytes during the contact with phytohaemagglutinin (T-lymphocytes stimulator) with people having or not having an oncological disease.

Principal possibility of attaining the goal by means of the device proposed was certified by the discovery of Cercek Boris and Cercek Lea (first from Manchester, England, and then California, USA) who have studied the phenomenon of destructuredness of a living cell body before the start of its division which (destructuredness) can be fixed by means of a special optic device permitting to measure the degree of polarized light depolarization. (see Rem Petrov “Me or not me”, Moscow, Russia, editing house “Molodaya Gvardia”, 1987, p. 216-217).

{(As is known, light polarization is the process of putting in order the oscillations of electric field intensity vector in light wave during light trespassing through some substances (during refraction) or during reflecting a light flow.

Polarizer is a substance/device serving for natural light transformation into plane-polarized one. http://www.b-i-o-n.ru/theory/elektromagnitnye-volny/poljarizacija-sveta)}.

Using the device mentioned the scientists have checked the action of phytohaemagglutinin—a strong stimulator of human T-lymphocytes. After some time (minimum 20 minutes) destructuredness of the cell body has been fixed, thus certifying lymphocytes activation and their readiness for multiplication.

It appeared that the mechanism of T-lymphocytes stimulation is different with healthy people and cancer patients.

The most important part of what was discovered by the Cercek couple is the fact that T-lymphocytes of cancer patients are activated during their contact with protein secreted by a malignant tumor.

The fact is that all cancer cells secrete specific proteins which are embryonic and specific for each kind of malignant tumor.

Being products of cancer cells living activity, these protein substances become targets for antibodies which destroy them.

But in reality it appears that the antibodies in this case actually guard cancer cells with the corresponding tragic outcome for the patient.

The importance of the proposed device for early cancer diagnostics is connected with exactly these circumstances (taking into account that neither Cercek couple, not anybody else after them have made this inventor's step which can lead to a medical result declared by the author of the proposed invention in the present application; it is confirmed by information from mentioned above Cerceks patents U.S. Pat. No. 5,552,292 dated 1994, U.S. Pat. No. 5,270,788 dated 1993 and also by decisions of other authors).

In mentioned above patent materials the author of proposed solution didn't find the confirmation of hospital success the solutions of the respected scientists in the sphere of discovery cancer at the initial stage and at the same time universality of their approach to discovery cancer irrespective of the oncological disease kind and the body organ targeted.

It's not look through also sufficient simplicity (and consequently also cost decrease) of measure for early diagnostics of cancer.

It allows make the conclusion about that the Cercek couple solutions have no essential advantages to the method using by the laboratory “Oncolab”.

The same sircumstance permits to the author of proposed solution to hope for realization of the declaring in present application advantages, which will be proved in visible future in a medical institution, that is in hospital practice.

Here, known to the author the solution according to the patent of Cercek couple, in fact relate to the basis properly cancer treatment (in future it will be possible to compare with other approach with author's proposed solution—with method of cancer treatment, showed in U.S. provisional application No. 61/404,213 from Sep. 30, 2010 and non-provisional application from Jul. 14, 2011) and, in author's opinion, yield to his problem solution on final result.

The device includes two polarization filters in the form of plates 1₁ and 1₂ embracing transparent element 2 for positioning the studies sample of organic substance (mixture of small portions of blood lymphocytes with phytohaemagglutinin), positioned parallel and with mutually perpendicular direction of polarization vectors between light source 3 and microscope ocular 4 with photoelement 5 at the exit. (Lymphocytes are obtained by blood separation from blood samples taken from inspecting patients).

In this case element for positioning the sample forms a delivery unit (e.g. in the form of film which is rolled off the reel by a servomotor and going to reclamation-into an utilizator) for delivering the mixture of patient's blood lymphocytes 6 with phytohaemagglutinin 7 during 21-25 minutes from the moment of the said samples contact with phytohaemagglutinin which is coming from unit 8 for its delivery to each portion of blood lymphocyte sample at the entrance of unit 9 for delivering the studied sample of blood lymphocytes.

Hereby polarization filters. unit for blood lymphocyte samples delivery, unit for phytohaemagglutinin delivery and capacities 10 and 11 for delivery of blood lymphocytes samples and phytohaemagglutinin are positioned in thermostat 12 with working temperature of 36.5-37.5° C. for preventing blood coagulation.

When light goes through polarization filters (plates) in the microscope and is not fixed by the photoelement, i.e. when lymphocytes do not manifest the properties of polarized light depolarization, the presence of oncological disease in initial stage is stated irrespective of the kind of disease and organ targeted.

When light goes through polarization filters (plates), i.e. when lymphocytes manifest the properties of polarized light depolarization, the absence of oncological disease is stated.

Thus, the proposed device is novel, possesses an inventive step, is useful, industrially applicable and should be lawfully protected by a patent.

I claim:

1. A device for early diagnostics of cancer comprising
a microscope with light source at the entrance and a photoelement at the exit, as well as a transparent element for positioning an studied organic substance sample between them, characterized by including
two polarization filters in the form of plates embracing the said element for positioning the sample which are situated parallel and with mutually perpendicular direction of polarization vectors between the light source and the microscope ocular,
hereby the element for positioning the sample forms a unit for delivering the studied sample in the form of a mixture of small portions of human blood T-lymphocytes with phytohaemagglutinin during 21-25 minutes from the moment of the said samples contact with phytohaemagglutinin,
which is coming from the unit for its delivery to each portion of blood lymphocyte sample at the entrance to a unit for delivering the studied sample of blood T-lymphocytes,
hereby the said polarization filters, unit for blood T-lymphocyte samples delivery, unit for phytohaemagglutinin delivery and capacities for blood T-lymphocyte samples and phytohaemagglutinin are positioned into a thermostat with working temperature 36.5-37.5° C. for preventing blood coagulation,
with providing for early diagnostics of oncological disease irrespective of the kind of disease and organ targeted.
